(19) United States
(12) Patent Application Publication
(10) Pub. No.: US 2008/0077186 A1
(43) Pub. Date: Mar. 27, 2008

(54) HIGH PHRENIC, LOW CAPTURE THRESHOLD PACING DEVICES AND METHODS

(75) Inventors: Todd Thompson, San Jose, CA (US); Mark J. Zdeblick, Portola Valley, CA (US); Olivier Colliou, Los Gatos, CA (US); Benedict James Costello, Berkeley, CA (US); Marc Jensen, Los Gatos, CA (US); George M. Savage, Portola Valley, CA (US)

Correspondence Address: BOZICEVIC, FIELD & FRANCIS LLP;(PROTEUS BIOMEDICAL, INC), 1900 UNIVERSITY AVENUE, SUITE 200, EAST PALO ALTO, CA, 94303, US

(73) Assignee: Proteus Biomedical, Inc.

(21) Appl. No.: 11/734,617

(22) Filed: Apr. 12, 2007

Related U.S. Application Data
(60) Provisional application No. 60/793,295, filed on Apr. 18, 2006.
(60) Provisional application No. 60/807,289, filed on Jul. 13, 2006.

(51) Int. Cl. A61N 1/00

(52) U.S. Cl. 607/9

(57) ABSTRACT

Methods of highly selective cardiac tissue stimulation and devices for practicing the same, e.g., implantable segmented electrode devices, are provided. The methods and devices provide a previously unavailable high phrenic nerve capture voltage paired with a low pacing capture voltage threshold. The subject methods and devices provide a number of benefits. For example, patients who previously would have been required to have their resynchronization device turned off due to phrenic nerve capture will now be able to reap the benefits of resynchronization therapy.

### CROSS-REFERENCE TO RELATED APPLICATIONS

Pursuant to 35 U.S.C. § 119 (e), this application claims priority to the filing dates of U.S. Provisional Application Ser. Nos. 60/793,295 filed on Apr. 18, 2006 and 60/807,289 filed on Jul. 13, 2006; the disclosures of which are herein incorporated by reference.

This application is a continuation in part of application serial no. PCT/US05/046811 filed Dec. 22, 2005; which application claims priority to the filing dates of: U.S. Provisional Patent Application Ser. No. 60/638,692 filed Dec. 22, 2004; U.S. Provisional Patent Application Ser. No. 60/655,609 filed Feb. 22, 2005; U.S. Provisional Patent Application Ser. No. 60/751,111 filed Dec. 15, 2005 and titled “Fatigue Resistant IC Chip Connection”; and U.S. Provisional Patent Application Ser. No. 60/752,733 filed Dec. 20, 2005 and titled “Fatigue Resistant Coiled IC Chip Connection”; the disclosures of which applications are herein incorporated by reference.

### BACKGROUND

Cardiac rhythm management devices are implantable devices that provide electrical stimulation to selected chambers of the heart in order to treat disorders of cardiac rhythm. A pacemaker, for example, is a cardiac rhythm management device that paces the heart with timed pacing pulses. The most common condition for which pacemakers are used is in the treatment of bradycardia, where the ventricular rate is too slow. Atrio-ventricular conduction defects (i.e., AV block) that are permanent or intermittent and sick sinus syndrome represent the most common causes of bradycardia for which permanent pacing may be indicated. If functioning properly, the pacemaker makes up for the heart's inability to pace itself at an appropriate rhythm in order to meet metabolic demand by enforcing a minimum heart rate.

Pacemakers are usually implanted subcutaneously or submuscularly on a patient's chest and have leads threaded intravenously into the heart to connect the device to electrodes used for sensing and pacing. Leads may also be positioned on the epicardium by various means. A programmable electronic controller causes the pacing pulses to be output in response to lapsed time intervals and sensed electrical activity (i.e., intrinsic heart beats not as a result of a pacing pulse). Pacemakers sense intrinsic cardiac electrical activity by means of internal electrodes disposed near the chamber to be sensed. A depolarization wave associated with an intrinsic contraction of the atria or ventricles that is detected by the pacemaker is referred to as an atrial sense or ventricular sense, respectively. In order to cause such a contraction in the absence of an intrinsic beat, a pacing pulse (either an atrial pace or a ventricular pace) with energy above a certain pacing threshold is delivered to the chamber via the same or different electrode used for sensing the chamber.

Electrical stimulation of the heart through the internal electrodes, however, can also cause unwanted stimulation of skeletal muscle. The left phrenic nerve, which provides innervation for the diaphragm, arises from the cervical spine and descends to the diaphragm through the mediastinum where the heart is situated. As it passes the heart, the left phrenic nerve courses along the pericardium, superficial to the left atrium and left ventricle. Because of its proximity to the electrodes used for pacing, the nerve can be stimulated by a pacing pulse. The resulting involuntary contraction of the diaphragm can be quite annoying to the patient, similar to a hiccup.

A variety of different approaches have been developed in order to address the issue of unwanted phrenic nerve capture. For example, Published U.S. Application No. 20030065365 discloses a device which includes an accelerometer that is used to detect diaphragmatic or other skeletal muscle contraction associated with the output of a pacing pulse. Upon detection of diaphragmatic contraction, the device may be configured to automatically adjust the pacing pulse energy and/or pacing configuration.

There continues to be a need for the development of cardiac stimulation devices whose stimulatory output can be delivered in a highly controlled manner. Of particular interest would be the development of a lead which can provide a focused cardiac stimulation that is sufficiently large to provide the desired capture while at the same time produced in such a manner as to avoid phrenic nerve capture. The present invention satisfies this, and other needs.

### SUMMARY

Methods of highly selective cardiac tissue stimulation and devices for practicing the same, e.g., implantable segmented electrode devices, are provided. The methods and devices provide a previously unavailable high phrenic nerve capture voltage paired with a low pacing capture voltage threshold. The subject methods and devices provide a number of benefits. For example, patients who previously would have been required to have their resynchronization device turned off due to phrenic nerve capture will now be able to reap the benefits of resynchronization therapy.

Additionally, the low pacing capture voltage threshold achieved by the present invention has many important clinical and technical advantages. Selectivity of cardiac muscle capture is unprecedented as compared to previously available devices. The low pacing capture voltage allows the advantages of low energy consumption. Additionally, it brings patients who would be at too high a voltage level for safe or effective pacing into a range where they, too, can enjoy the benefits of resynchronization therapy.

In certain embodiments, the highly selective stimulation devices include segmented electrode structures made up of two or more electrodes positioned close to each other, where the electrodes can be individually activated. In certain embodiments, the segmented electrodes include at least one cathode and at least one anode from which highly localized stimulatory energy may be produced. The electrode components of each segmented electrode can be individually activated. In certain embodiments, the segmented electrodes include an integrated circuit electrically coupled to two or more electrodes, where each electrode can be individually activated. Also provided are implantable devices and systems, as well as kits containing such devices and systems or components thereof, which include the segmented electrode structures.

Aspects of the invention include electrodes that are segmented, e.g., to provide better current distribution in the tissue/organ to be stimulated. In such embodiments, the segmented electrodes are able to pace and sense independently with the use of an integrated circuit (IC) in the lead, such as a multiplexing circuit, e.g., as disclosed in PCT Application No. PCT/US2005/031559 titled “Methods and Apparatus for Tissue Activation and Monitoring” and filed on Sep. 1, 2005; the disclosure of which is herein incorporated by reference. The IC allows each electrode to be addressed individually, such that each may be activated individually, or in combinations with other electrodes on the medical device. In addition, they can be used to pace in new and novel combinations with the aid of the multiplexing circuits on the IC.

Aspects of the invention further include methods of using the addressable segmented electrode structure of the implanted medical device, e.g., to deliver electrical energy to the subject, e.g., in a highly specific manner that results in a high phrenic nerve capture threshold but low cardiac tissue capture threshold. In certain embodiments, at least a first of the electrodes is connected to a first conductive member and a second of said electrodes is connected to a second conductive member. In certain embodiments, the method includes not activating at least one of the electrodes, such as activating only one of said electrodes. In certain embodiments, the method further includes determining which of the electrodes to activate. In certain embodiments, the method further includes sequentially activating the electrodes. In certain embodiments, the method includes minimizing power consumption. In certain embodiments, the method includes activating the electrodes in manner sufficient to not stimulate the phrenic nerve. In certain embodiments, the method includes activating at least one of the electrodes of the structure to sense electrical potential in said subject.

Aspects of the invention further include systems and kits that include an implantable addressable segmented electrode structure according to the invention.

### BRIEF DESCRIPTION OF THE FIGURES

FIG. 1 shows the configuration of segmented electrode structure that includes four electrodes (e.g., quadrant electrodes) positioned around an IC in an aligned configuration according to an embodiment of the invention;

FIG. 2 provides a view of a medical device cross section that is not round, according to an embodiment of the invention;

FIGS. 3A to 3C provide views of a simplified version of the device shown in FIG. 22, where the lead and electrodes are incorporated into a single piece;

FIG. 4 provides a view of an approach to assembly of a structure according to an embodiment of the invention;

FIG. 5 provides a view of an approach to assembly of a structure according to an embodiment of the invention;

FIG. 6 shows an IC connected to a multiplicity of electrodes, e.g., in a quadrant electrode configuration, according to an embodiment of the invention;

FIG. 7 describes an IC attached to the electrodes in a helix configuration supported by a polymer; according to an embodiment of the invention;

FIG. 8 describes an IC connected to electrodes dispersed along the length of a medical device, according to an embodiment of the invention;

FIG. 9 illustrates an overall view of the completed assembly that includes spring connectors, according to an embodiment of the invention;

FIG. 10 provides a depiction of a cardiac resynchronization therapy system that includes one or more hermetically sealed integrated circuits coupled to lead electrodes according to an embodiment of the invention.

FIG. 11 provides a table showing experimental data of Phrenic capture which is 20× greater than the cardiac capture threshold.

FIG. 12 provides a table showing experimental data of the ratio of the phrenic nerve capture voltage to the cardiac capture voltage.

### DETAILED DESCRIPTION

As summarized above, the present invention provides methods and devices for highly specific tissue, e.g., cardiac tissue, stimulation. Aspects of the invention include segmented electrode devices, as well as methods for making and using the same. These devices provide a previously unavailable high phrenic nerve capture voltage paired with a low pacing capture voltage threshold. Patients who previously would have been required to have their resynchronization device turned off due to phrenic nerve capture now are able to reap the benefits of resynchronization therapy. Selectivity of cardiac muscle capture by the inventive device is unprecedented as compared to previously available devices.

Additionally, the low pacing capture voltage threshold achieved by the present invention allows the advantages of low energy consumption. Additionally, it brings patients who would be at too high a voltage level for safe or effective pacing into a range where they, too, can enjoy the benefits of resynchronization therapy.

Embodiments of the devices include segmented electrode structures of two or more closely spaced electrodes. In certain embodiments, the segmented electrode structures are made up of an integrated circuit electrically coupled to two or more electrodes, where each electrode can be individually activated. Also provided are implantable devices and systems, as well as kits containing such devices and systems or components thereof, which include the segmented electrode structures. Embodiments of the invention are particularly suited for use in multiplex lead devices, as these embodiments can have appropriate dimensional variety of IC chips and their accompanying electrodes with internal connections, and conductive connections with structures are robust to impart fatigue resistance to the structures.

In further describing aspects of the invention, methods of highly specific tissue stimulation and devices, e.g., that include segmented electrode structures, that find use in practicing the same, are reviewed first in greater detail, both generally and in terms of figures of certain embodiments of the invention. Next, embodiments of devices and systems, such as implantable medical devices and systems, that include the segmented electrode structures of the invention are described. Also provided is a description of kits that incorporate aspects of the invention.

High Phrenic, Low Capture Threshold Tissue Stimulation Methods and Devices

As summarized above, the methods of the invention are highly specific tissue stimulation methods, e.g., highly specific cardiac tissue stimulation. As such, the invention includes methods of focused cardiac tissue stimulation. By focused cardiac tissue stimulation is meant that electrical stimulation is generated from an electrode structure in an asymmetric directional manner from the electrode structure, such that the electrode structure does not provide symmetrical electrical stimulation to the same extent into all tissue surrounding the electrode structure. In certain embodiments, focused stimulation arises from a bipolar electrode structure, e.g., from an electrode structure having at least one anode and at least one cathode which are sufficient proximal to each other that, upon application of a suitable stimulatory current, an electrical stimulation is produced in the tissue that is contacted by the anode and the cathode. As the stimulations of the subject methods are selective, they have a high selectivity ratio, where selectivity ratio is determined by the formula:

Selectivity=unwanted nerve capture voltage/desired tissue capture voltage. In certain embodiments, the selectivity ratio of the subject methods is about 5 or higher, such as about 10 or higher and including about 15 or higher, e.g., 20 or higher.

Where the methods are methods of selective cardiac tissue stimulation with respect to the phrenic nerve, selectivity as determined using the following formula:

Selectivity=phrenic nerve capture voltage/cardiac capture voltage is about 5 or higher, such as about 10 or higher and including about 15 or higher, e.g., 20 or higher.

The selective stimulation feature of the subject methods also provides for embodiments of tissue stimulation in which the amount of voltage needed for effective capture is less than that employed in methods where tissue is not selectively stimulated. For example, in certain cardiac tissue stimulation methods, effective cardiac capture is achieved with voltages of about 10 volts or less e.g., about 5 volts or less, such as about 1.5 volts or less, including about 0.50 volts or less, such as about 0.25 volts or less.

Where the tissue that is stimulated in the subject methods is cardiac tissue, embodiments of the methods of cardiac tissue stimulation may be characterized as high phrenic nerve capture threshold, low cardiac tissue capture threshold methods. In these embodiments, cardiac tissue is stimulated in a manner such that the capture threshold for the phrenic nerve is significantly higher than the capture threshold for the cardiac tissue, e.g., about 5 times or more higher, such about 10 times or more higher and including about 20 times more or higher. In certain embodiments, the capture of the phrenic nerve only occurs with activation energies of about 3 to about 18 volts or higher, such as about 10 to about 17 volts or higher, including about 15 volts or higher.

Where desired, the methods may include a step of obtaining phrenic nerve capture data and employing this data in the selective tissue stimulation. For example, a sensor can be employed to detect phrenic nerve capture, and the resultant data employed to set or more modify the cardiac stimulation parameters of focused cardiac stimulation. The sensor may be present in the same lead or a different lead from the cardiac stimulation lead. Any convenient sensor may be employed. The sensor could be an electrical sensor if it is on the diaphragm or near the phrenic nerve or it could be a motion sensor or a mechanical motion sensor on the lead. Examples of suitable sensors include pressure sensors, strain gauges, accelerometers, acoustic sensors, where the sensors can be orientated anywhere along the lead or independently on another lead placed on the diaphragm.

In certain embodiments, feedback regarding phrenic nerve capture or lack thereof is provided so that if one is automatically repositioning electrodes the box can have a feedback mechanism and the circuit can make sure that it does not choose an inappropriate electrode that would cause phrenic stimulation. In addition, during the initial programming of the device it could provide feedback that would be sub-threshold or tactile threshold for the clinician when he is observing the patient or possibly also for the patient.

In other embodiments, data regarding phrenic nerve capture, e.g., from distinct devices associated with the diaphragm, such as a diaphragm lead, can be employed. Any convenient method of communicating the data from the diaphragm specific lead to the controller of the pacing lead may be employed, such as an RF or other suitable communication protocol.

As such, the phrenic nerve capture device could be inside the cardiac stimulation lead or associated with a deminimus ASIC chip or it could be a separate packaged assembly inside the lead and not exposed.

One can evaluate for a correlation between pacing pulses and EMG signals around diaphragm or phrenic nerve signals.

Another suitable protocol for testing for phrenic nerve capture is to use non-cardiac tissue pace inducing pulses, such as pulses at a higher frequency, at a different rate that the pace rate, e.g., slower than a cardiac pacing rate, or a different series of wave forms to test for phrenic capture independently of pacing. Alternatively, test pulses during the heart's refractory period may be generated. Such protocols may employ an external communicating device that could be positioned on the outside of the patient that would detect the higher frequency motions and then relay that to either the ICD in the person's chest or the computing device in the person's chest or the computer when this is going through programming. This device could also be attached so that if the pacing parameters are changed during an exercise or a stress test this could provide feedback during an exercise or stress test assuming the frequency of the vibrations would be detectable when it is overlaid on top of any kind of motion and this could be used during the night to monitor a patient over a period of days with an external device that would provide this detection and this device could be internally implanted. This device could be either attached through a lead or have an antenna and have radio frequency communication that would detect phrenic capture. This device would evaluate at the data set for the data from the different sensors so the data change of interest would be the data change that happened concurrently with pacing pulses. That would include both pressure changes and motion changes and, where desired, electrical pacing on a diaphragm on the surface of the diaphragm or near the diaphragm. So this device could also be an adhesively applied patch that would be applied to the patient over a period of from 1 hour to 24 or 48 hours. The device need not be continuously powered, but may be powered only during times when change is occurring. So if the ICD thinks it is about ready to try a different pacing location then one could turn on the sensor just to get feedback about phrenic nerve capture. Where desired, this sensor would be running for a period of time to catch several breath cycles do to the erratic nature of the capture of the phrenic nerve.

The above described methods of detecting phrenic nerve capture and employing the capture data in pacing are merely representative. The obtained phrenic nerve capture data may be employed in a number of different ways, such as in the initial determination of a pacing protocol (such as which electrodes of a segmented electrode structure to activate, the voltage to employ, etc.), in the modification of an existing pacing protocol, etc. In certain embodiments, the feedback may be open loop, such that phrenic nerve capture data is evaluated by a health care practitioner. The data may be provided in terms of a safety factor, e.g., ratio of heart capture threshold to phrenic nerve capture threshold during implant. As desired the health care practitioner may then set pacing parameters based on the phrenic nerve capture data. In yet other embodiments, the feedback is closed loop, such that a pacing protocol is automatically adjusted in response to the obtained phrenic nerve capture date, e.g., by a processor in an ICD or even by a processor in a chip that is part of a segmented electrode structure. In practicing the subject methods, any convenient electrical stimulation device that can provide for the selective tissue, e.g., cardiac tissue, stimulation may be employed. One type of device that may be employed in the subject methods is a segmented electrode device, i.e., a device that includes a segmented electrode structure. As summarized above, a segmented electrode structure is an electrode structure made up of two or more distinct electrode elements positioned proximal to each other, e.g., on a support such as a lead, where the electrode elements can be activated in a manner sufficient to provide for selective tissue stimulation, e.g., as described above. The segmented electrode structures may be configured to produce bipolar electrical stimulation, in which one of the electrode elements of the structure acts as the anode and the other electrode element(s) acts as the cathode, such that an electrical field is generated between the electrode elements which provides focused stimulation to the tissue in contact with the segmented electrode structure.

In certain segmented electrode embodiments, the methods include “pacing” between electrode elements of the same band, i.e., between two or more of the electrode components of the same segmented electrode structure. As such, these embodiments are distinguished from non-segmented electrode applications in which pacing may occur between two different bands on a lead, since the embodiments of the subject invention may be characterized as intraband pacing embodiments, as opposed to interband pacing embodiments.

In certain embodiments, the area of the anode is greater than the area of the cathode, e.g., by a factor of about 3:1 or more, such as by a factor of about 10:1 or more, including by factor of about 15:1 or more. In certain embodiments, the anode element(s) may surround or circumscribe the cathode elements. In yet other embodiments, the anode elements may be inter-digitated with the cathode elements.

The segmented electrode structures may vary considerably, so long as the different electrode elements are sufficiently proximal to each other to generate the desired electric stimulation. Distances between the electrode structures may vary, where in certain embodiments, the distances are about 1000 μm or less, such as about 500 μm or less, and in certain embodiments range from about 5 μm to about 1000 μm, such as from about 50 μm to about 500 μm and including from about 100 to about 300 μm, e.g., about 200 μm.

Where the segmented electrode structure is present on a lead or analogous carrier, the electrode structure may be conductively coupled to an elongated conductive member, e.g., to provide for communication with a remote structure, such as a remote controller, e.g., which may be present in a structure which is known in the art as a “can.” As such, in certain embodiments, the segmented electrode structures are electrically coupled to at least one elongated conductor, which elongated conductor may or may not be present in a lead, and may or may not in turn be electrically coupled to a control unit, e.g., that is present in a pacemaker can. In such embodiments, the combination of segmented electrode structure and elongated conductor may be referred to as a lead assembly.

In certain embodiments, each electrode element of the segmented structure may be coupled to its own conductive member or members, such that each electrode element is coupled to its own wire. In these embodiments the structure or carrier, e.g., lead, on which the structure is present may be torqueable, such that it can be turned during and upon placement of the lead so that upon activation, the electrode elements produce stimulation in the desired, focused direction.

In yet other embodiments, the electrode elements of the structure are present on a multiplex lead, such that two or more disparate electrode structures are coupled to the same lead or leads. A variety of multiplex lead formats are known in the art and may readily be adapted for use in the present devices. See e.g., U.S. Pat. Nos. 5,593,430; 5,999,848; 6,418,348; 6,421,567 and 6,473,653; the disclosures of which are herein incorporated by reference. Of particular interest are multiplex leads as disclosed in published U.S. Patent application no. 2004-0193021; the disclosure of which is herein incorporated by reference.

Of interest are structures that include an integrated circuit (IC) electrically coupled (so as to provide an electrical connection) to two or more electrode elements. The term “integrated circuit” (IC) is used herein to refer to a tiny complex of electronic components and their connections that is produced in or on a small slice of material, i.e., chip, such as a silicon chip. In certain embodiments, the IC is a multiplexing circuit, e.g., as disclosed in PCT Application No. PCT/US2005/031559 titled “Methods and Apparatus for Tissue Activation and Monitoring” and filed on Sep. 1, 2005; the disclosure of which is herein incorporated by reference. In the segmented electrode structures, the number of electrodes that is electrically coupled to the IC may vary, where in certain embodiments the number of 2 or more, e.g., 3 or more, 4 or more, etc., and in certain embodiments ranged from 2 to about 20, such as from about 3 to about 8, e.g., from about 4 to about 6. While being electrically coupled to the IC, the different electrodes of the structures are electrically isolated from each other, such that current cannot flow directly from one electrode to the other. In these embodiments, the lead need not be torqueable, since the desired focused stimulation can be achieved through selective activation of electrodes.

As the structures are implantable, that may be placed into a physiological site and maintained for a period of time without substantial, if any, impairment of function. As such, once implanted in or on a body, the structures do not deteriorate in terms of function, e.g., as determined by ability to activate the electrodes of the structure, for a period of at least about 2 or more days, such as at least about 1 week, at least about 4 weeks, at least about 6 months, at least about 1 year or longer, e.g., at least about 5 years or longer. As the electrodes of the subject segmented electrode structures of these embodiments are addressable, they can be individually activated. As such, one can activate certain of the electrodes of the structure while not activating others, e.g., in manner such that electrical stimulation can be delivered from one or more of the electrodes of the structure, but not all of the electrodes in the structure, where in certain embodiments only a single electrode of the structure is activated at any given time. As another example, one can activate one electrode in such a way that it conducts electric potentials from nearby tissue to the electric circuitry. In some embodiments, activate may further comprise electrically connecting an electrode to a conductor, such as a bus conductor, for stimulation, voltage sampling, or other purposes. In certain embodiments, the elongated conductive member is part of a multiplex lead, e.g., as described in Published PCT Application No. WO 2004/052182 and published U.S. Patent application no. 2004-0193021, the disclosure of which is herein incorporated by reference.

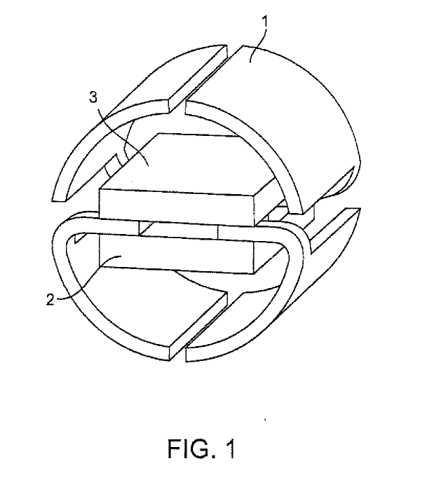

In certain embodiments, the electrodes of the segmented electrode structures are electrically isolated from each other, and may be circumferentially arranged around an IC to which they are conductively coupled. An example of such an embodiment is shown in FIG. 1, where four separate electrodes are electrically coupled to a single IC in what is referred to herein as a quadrant electrode configuration. As can be seen in the figure, the electrodes are circumferentially arranged about the central IC. In the embodiment depicted in FIG. 1, the segmented electrodes are arranged about the IC to form a cylinder shaped structure, which is suited for use in many different medical devices, as illustrated below. However, the structure may have any convenient shape, such as a flattened cylinder, oval shape, or other shape, as desired. In certain embodiments, the electrodes of the segmented electrodes are aligned, e.g., having one edge, e.g., the proximal edge, of each electrode shares a common plane as shown in FIG. 1. In yet other embodiments, the different electrodes may be present in an offset configuration, for example in a staggered configuration, e.g., as shown in FIG. 8. By “staggered” is meant that at least one of the edges of the electrodes does not share a common plane. In yet other embodiments, the electrodes may have an interdigitated arrangement.

In embodiments of the invention, the structures are dimensioned to be placed inside a lead, e.g., cardiovascular lead, epicardial lead, left ventricular lead, etc., or implant. By “dimensioned to be placed inside of a lead or implant” is meant that the structures have a sufficiently small size (i.e., form factor) such that they can be positioned inside of a lead or implant. In certain embodiments, the hermetically sealed structures have a longest dimension, e.g., length, width or height, ranging from about 0.05 mm to about 20 mm, such as from about 0.2 mm to about 5 mm, including from about 0.5 mm to about 2 mm. Accordingly, embodiments of the structures allow the practical development of miniaturized, implantable medical devices for days, months, and even years of practical, reliable use.

Embodiments of the invention include implantable fatigue resistant structures. In such embodiments, at least the IC and electrode components of the segmented structure, for example, the IC, electrode and conductor components of a lead assembly, are electrically coupled to each other in a manner that imparts fatigue resistance to structure and/or lead assembly that contains the structure. This fatigue resistance ensures that the structures can survive intact (i.e., without substantial, if any, breakage of the connections between the integrated circuit and electrode(s) components of the structure) in an in vivo environment, such as in a physiological environment in which they are in contact with blood, and/or tissue. Because the structures are implantable, the implantable structures are structures that may be positioned in or on a body and function without significant, if any, deterioration (e.g., in the form of breakage of connections, such as determined by function of the segmented electrode structure) for extended periods of time. As such, once implanted, the structures do not deteriorate in terms of function, e.g., as determined by function of an integrated circuit and electrodes coupled thereto of the structure, for a period of at least about 2 or more days, such as at least about 1 week, at least about 4 weeks, at least about 6 months, at least about 1 year or longer, e.g., at least about 5 years or longer.

Aspects of the invention include one or more features that impart fatigue resistance to the subject segmented electrode structures. Fatigue resistance imparting features include, but are not limited to electrical connections between components, e.g., electrodes, IC, elongated conductive members, that minimize mechanical stress between the connected components. For example, flexible conductive connectors of a variety of different materials and/or configurations are employed in certain embodiments of the invention, as described in greater detail below. In yet other embodiments, liquid conductive connectors of a variety of different materials and/or configurations are employed which provide for a high degree of freedom of movement between connected components, as described in greater detail below. In yet other embodiments, non-bound conductive connectors of a variety of different materials and/or configurations, e.g., rigid spheres, coils/springs, etc., are employed which provide for a high degree of freedom of movement between connected components, as described in greater detail below. In these embodiments, “non-bound” means that the connector is not physically immobilized on a region of the connected component, but is instead capable of moving across a surface of the connected component, at least in some plane, while still maintaining the conductive connection. Of interest are the structures disclosed in PCT application serial no. US2005/046811; the disclosure of which is herein incorporated by reference.

In certain embodiments, the IC component of the structures is hermetically sealed, e.g., it is present in a hermetically sealed structure that includes a hermetically sealed volume which houses one or more ICs. Aspects of the invention include hermetically sealed ICs that include: an in vivo corrosion resistant holder having at least one conductive feedthrough; and a sealing layer; where the sealing layer and the holder are configured to define a hermetically sealed volume, e.g., in which one or more ICs is present. Such hermetically sealed structures are further described in copending PCT patent application serial no. PCT/US2005/046815 titled “Implantable Hermetically Sealed Structures,” and filed on even date herewith, the disclosure of which is herein incorporated by reference.

The advantages of the present innovation of separately addressable segmented, e.g., quadrant electrodes, are many fold. Because the distribution of electrical potential (e.g., cardiac pacing pulse) can be directed, a great flexibility is provided in clinical applications. For example, by selectively activating one or more of the electrodes of the segmented structure, electrical current can be directed to only that tissue that needs to be excited, thereby avoiding excitation of tissue that is not desired to be excited. This feature provides multiple benefits. For example, in prior art methods, a left ventricular pacing electrode would typically have to be disabled, and the cardiac resynchronization therapy (CRT) intervention terminated, if phrenic nerve capture by the electrode caused the patient to suffer a diaphragmatic spasm with each discharge. By the careful electrode selection to control the directionality of electric current provided by the present invention, capture of the phrenic nerve can often be avoided, while appropriate levels of cardiac stimulation are maintained.

In addition, any given electrode can have a small surface area and still adequately excite the tissue that needs to be excited. For example, electrodes having a surface areas ranging from about 0.1 mm² to about 4.0 mm², such as from about 0.5 mm² to about 3.0 mm² may be employed. Despite their small surface area, excitation of that tissue that needs to be excited is achieved. When the segments are distributed around the circumference of a pacing lead, excitable tissue will be contacted regardless of the rotational orientation of the device in the vessel. With the reduced surface area of the electrode segments, the impedance is larger than that of a ring electrode of equal axial length thereby reducing the current drain on the pacemaker, which can lead to improved longevity of the device. Experimental data from epicardial left ventricular pacing with a four segment electrode structure have demonstrated an eight-fold difference in capture threshold between those segments that are in contact with cardiac tissue and those which are not. As such, with appropriate segmented electrode configuration, capture threshold differences of ten-fold or more may be achieved. The capture threshold, as defined as the minimum voltage that initiates excitation of the heart tissue, is directly proportional to power consumption of a pacemaker.

The inventive use of separately addressable quadrants on a multiple electrode leads allows a number of other clinical advantages. In many cases, the present invention allows patients who would be non-responsive using prior art devices to become responsive to treatment. For example, multiple potential excitation positions along the lead allows for selection in real time of the most advantageous pacing, without requiring repositioning of the lead. Synergistic use of multiple points of stimulation are also available (simultaneously or sequentially), again without any further lead positioning. Currently available techniques require difficult and often unsuccessful repositioning of the lead when an effective excitation position is not achieved. Because of difficulties in variations of anatomical features, and limitations in time available for repositioning, often results are sub-optimal or poor. Additional advantages include the ability to achieve fine measurement of conduction velocity in different axes.

In addition, in electrical tomography embodiments such as those described in POT Patent Application No. US2005/036035 titled “Continuous Field Tomography” filed Oct. 6, 2005, the subject structures permit calibration of local electric field gradients to improve accuracy in synchrony quantification and possibly enable absolute measurements (e.g., stroke volume, ejection fraction, etc.). In electrical tomography applications, applied electric fields are distributed in a curvilinear fashion within the body. Knowing the local field gradient in the region of interest (e.g., a cardiac vein overlying the LV) permits absolute determination of the local relationship between electrical distance (gradient) and physical distance.

Embodiments of the segmented electrode structures may include one or more of the above features, or others. In further describing the invention, embodiments of the structures are now reviewed in greater detail in terms of the figures.

As mentioned above, FIG. 1 provides a representation of a segmented electrode structure according to an embodiment of the invention. Cardiac pacing electrodes of the present invention may vary, and in certain embodiments range from about 0.1 to about 4 mm² in area, e.g., about 1.5 mm² in area. The electrodes can be positioned relative to the IC in a variety of different formats, e.g., circumferentially around the IC and/or the body of a lead, or they could be distributed longitudinally along the length of the lead body, extending from the connection from the IC or they could be arranged in a pattern that improves tissue contact or that facilitates measurement of local electrical field gradients.

A configuration of electrodes around the IC according to an embodiment of the invention, which is referred to herein as a quadrant electrode embodiment, is shown in FIG. 1. The four electrodes 1 are distributed around the IC in a circumferential pattern. Electrode 1 is shown as a solid surface but it may have a finer scale pattern formed into the surface that improves the flexibility of the electrode. IC chip 2 is hermetically sealed and provides a multiplexed connection to conductors in the lead (not shown in this figure). Optionally, top cap 3 is bonded to the integrated circuit. Cap 3 is a component that helps support the electrode to integrated circuit connection. Cap 3 may contain additional circuits or sensors. In certain embodiments, this assembly is incorporated into a flexible material, e.g., polymeric material, to form the body of the device. The device may be round or some other shape best suited to the particular location in the body where it is intended to be deployed.

The materials of construction of the conductive members, e.g., electrodes, for use with the presently described ICs may be primarily platinum, or platinum alloy, including platinum 5% iridium, platinum 10% iridium, or platinum 20% iridium. Additional appropriate platinum alloys include, but are not limited to: platinum 8% tungsten, platinum nickel, and platinum rhodium. The alloy could also be gold tin with gold 20% tin alloy. An additional material for the electrode of the present invention can be titanium. The titanium could be plated with platinum or platinum alloys previously described. Corrosion resistant alloys can also be deposited by RF Sputtering, electron beam vapor deposition, cathodic arc deposition, or chemical vapor deposition, among other methods. In addition to titanium, base electrode materials can include stainless steel, e.g., 316SS, or cobalt based super alloys, e.g., MP35N, or tantalum. The electrode can also be electroformed. Of interest are the electrode materials and methods of fabrication, disclosed in PCT application serial no. US2005/046811; the disclosure of which is herein incorporated by reference. Embodiments of the invention include the use of flexible conductive connectors between different components and/or electrode structures.

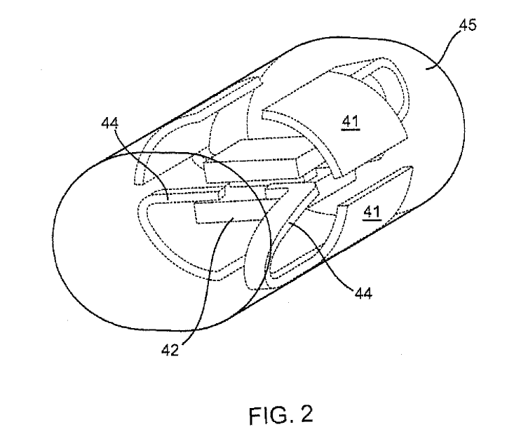

Of interest are the flexible electrode connectors and/or structures disclosed in PCT application serial no. US2005/046811; the disclosure of which is herein incorporated by reference. FIG. 2 provides an embodiment of a segmented electrode structure with flexible electrode connectors. In FIG. 2 flexible members 44 connect curved planar electrodes 41 to an IC 42 is shown. The stress applied to the IC is reduced by increasing the amount of elastic strain the member can withstand, e.g., using materials and/or configurations as described above. In FIG. 2, the fatigue resistant IC/electrode structure is present in a lead body 45, and the outer curved surface of the electrodes 41 matches the configuration of the lead body.

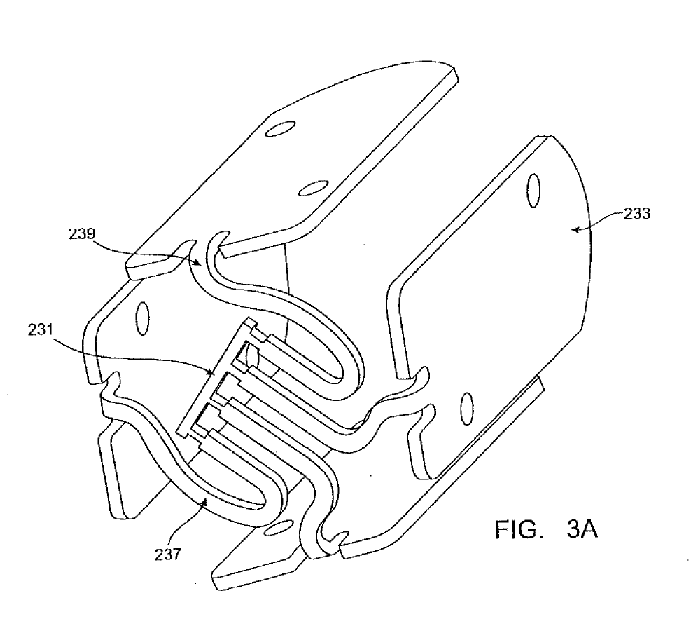

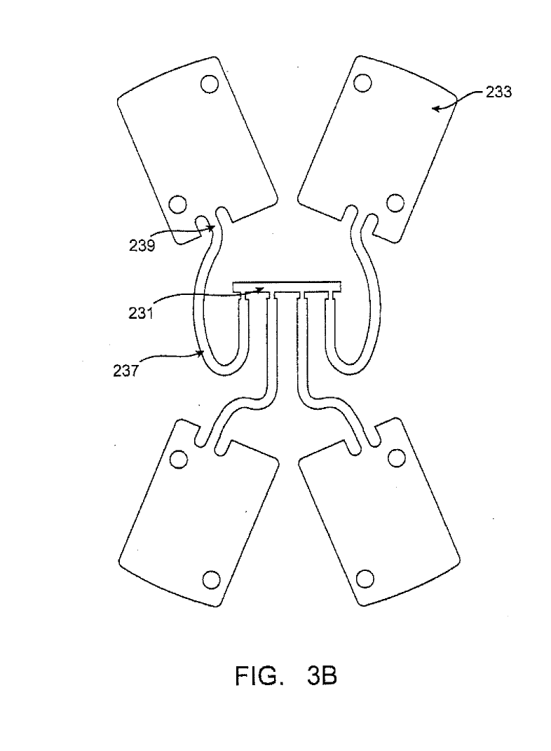

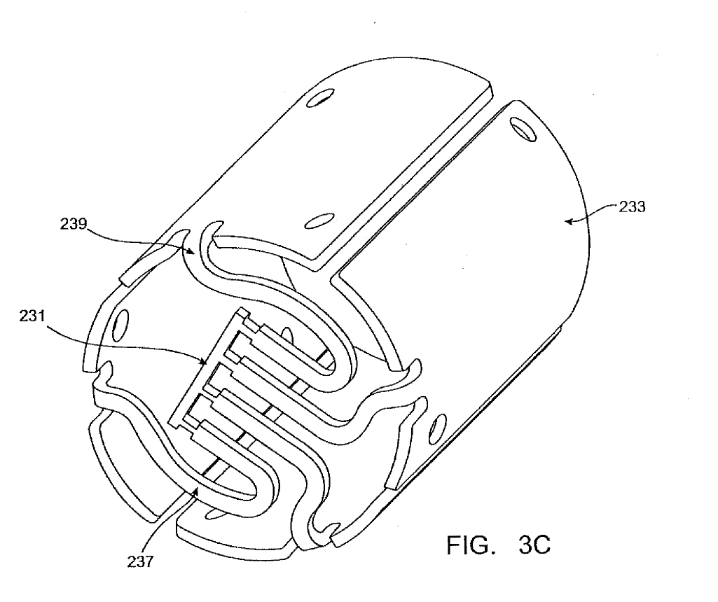

FIGS. 3A to 3C provide a view of an embodiment of the present invention. In FIG. 3A, the lead frame and four electrodes 224 are incorporated into a single piece via legs 237. The manufacturing process to produce the construct shown in FIG. 3A is simply accomplished by bending the electrodes down with relief 239. Sacrificial bar 231, supports the IC chip prior to full assembly. Sacrificial bar 231 keeps the assembly stable during the chip attachment step.

The assembly process for the inventive embodiment in FIG. 3A allows the whole device to exist on a single plane until the final stages of manufacture, as shown in FIG. 3B. The final manufacturing stage is when all four electrodes 233 are first bent down at juncture (i.e., relief) 239. Juncture 239 may be provided with triangular relief cutouts to provide for a smoother, less brittle connection to four electrodes 233. The final step in molding is shown in FIG. 3C where four electrodes 233 are each bent around their long axes to match the curvature of the lead body.

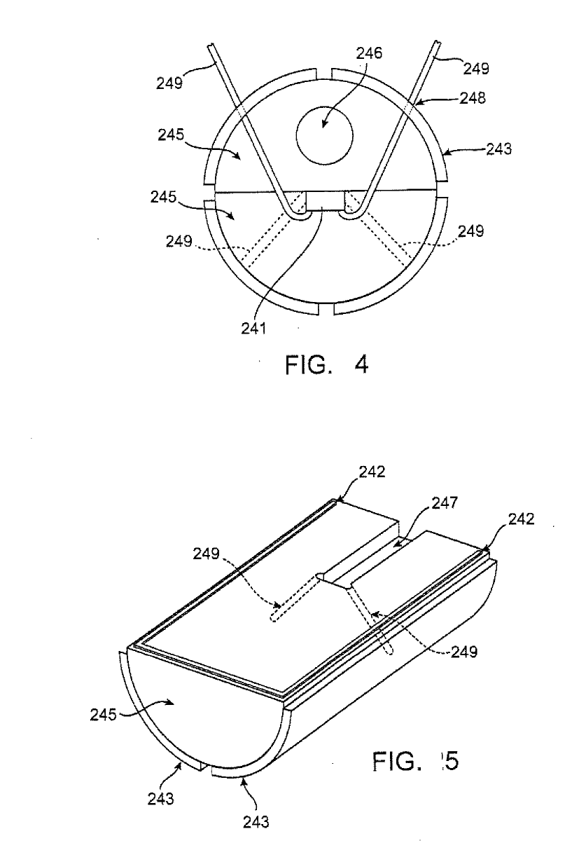

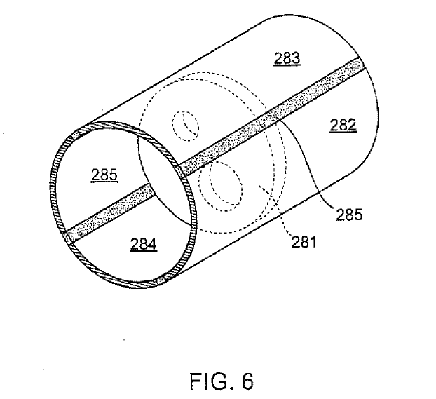

FIGS. 4 and 5 show a different approach to assembly. In this model, the IC chip is fitted into rectangular notch 247. Conductive vias 249 run out of rectangular notch 247 to carry the signal from the IC chip to the outside world. This embodiment of the present invention provides a way to seal the IC chip and provide attachments all at the same time. The IC chip within the cylinder contacts pads to make a connection to vias 249. The construct includes PEEK body 245. PEEK is a material which has a high-temperature melting point, allowing for soldering and other manufacture protocols. Rectangular notch 247 stabilizes the chip. Four conductive vias 249 are provided, which could be wires. In FIG. 4, four conductive vias 249 are provided. This design embodiment provides a method to seal the IC chip and provide attachments in a single step. Contact pads are provided on the IC chip that are aligned in one of the half-cylinder sections. This assembly provides a simple way to manufacture the inventive device. When PEEK is molten, it has very good adhesive properties which are exploited in one embodiment of the present invention. During manufacture, the PEEK is melted into the platinum electrodes 243. Two halves of the assembly, each a half cylinder, are manufactured as subassemblies.

The IC chip 241 is placed into rectangular notch 247. For an ultrasonic welding approach, a raised floss is provided. The sacrificial material 242 provides a good, fluid-tight seal when the two halves are aligned and welded together. This approach is useful to speed the assembly process, because the subassembly will be molded to have the vias and leads 249.

The IC chip is placed into the in rectangular notch 247 in the cylinder sub-structure half that will be place over the top of the full assembly. The two aligned halves are held in a clamshell type fixture, clamping the two halves together. Ultrasonic energy is applied, which melts the plastic together.

Sacrificial material 242 is engineered to be sacrificial, that is these pieces are designed to melt. Alternately, sacrificial material 242 can be placed to fully encircle or be placed inside rectangular notch 247. As a result, the whole construct is a sealed end, providing maximum hermeticity protection.

Alternately, an opening can be provided. The advantage to having an opening, at some point in the structure, is a place to pass through the power leads to the chip as may be desired. To provide stronger hermeticity protection in this case, it is possible to encapsulate the entire final structure. In the final stages of assembly, the wires have been passed through these vias 248 in FIG. 4. At this stage, the various components can be laser or resistance welded into place. The end of 249 just falls off. Guidewire lumen 246 is shown for orientation to the final device.

The fatigue resistant IC chip connections and assembly methods of the these and other embodiments described herein allow the practicable reproducible production of an IC chip package and attachment design which is uniquely scalable to the necessary dimensions for many medical device applications, such as, but not limited to, intracardiac and intraocular devices, e.g., as reviewed below. The present invention provides for an entire medical device which has the capacity to be scaled to the size of currently available chip-packages alone. This unique miniaturization of a device with robust qualities provides the clinician medical devices of unprecedented applications in their diagnostic and therapeutic armamentarium.

The inventive constructs and assembly methods provide means to get to the body with as short a path as possible from the chip. An important aspect of the present inventive fatigue resistant IC chip connection assembly methods provides very quick accesses or connects to the IC chip. It also provides very quick accesses or connects to the output of the chip to the body, or the chip to a package, or to a circuit or other device before it goes to the body. Though these multiply improved segments of the overall device, the invention allows a means to get to the body with a short as path as possible from the chip.

FIG. 6 shows IC chip connected to a multiplicity of electrodes, i.e., 281, 282, 283 and 284, where the electrodes are arranged in a quadrant configuration. The electrodes are connected to IC chip by solder 285 in this representation. However, other electrical connection methods are useful within the scope of this design. The electrodes are sized and positioned based on clinical requirements. This configuration allows a unique mass production method for the chip. The electrodes are embedded in an extended cylindrical shape. The surface is then polished, and the face cut.

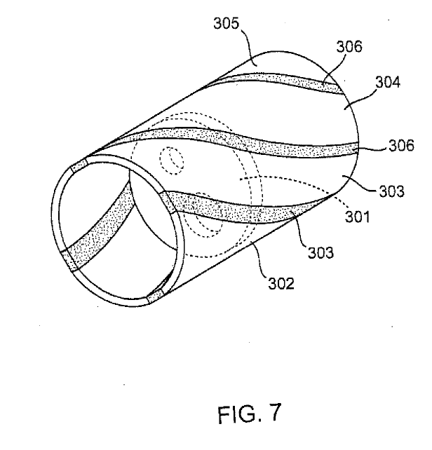

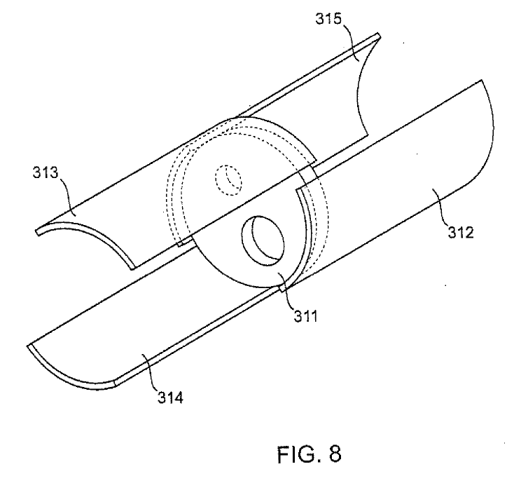

FIG. 7 describes IC chip 301 that is attached to electrodes 302, 303, 304 and 305. The electrodes are supported by polymer 306. The polymer 306 can be PEEK, PEKK, polyamide, ETFE, urethane, or other suitable material. The material may also be a ceramic material, alumina, silicon carbide or other suitable material. Embedding the electrodes in this manner provides many advantages, such as securing them in place, protecting them against possible biological fluid challenges, and providing a flexible support to cushion against impact forces. The electrodes reconfigured in a helix in this representation, but can take other forms as well.

FIG. 8 describes IC chip 311 connected to electrodes 312, 313, 314 and 315 that are dispersed along the length of the medical device. In this inventive configuration, two of the electrodes 312, 315 are more distal from IC chip 311, while two of the electrodes 313,314 are more proximal from IC chip 311. This form of configuration provides the opportunity for larger features to be accommodated within the medical device. It also disperses the strain, and provides for more flexibility than might otherwise be available. Additional, flexibility can be customized along the length of the device to provide optimum variable rigidity, such as may be required when accessing the coronary sinus.

FIG. 49 provides a depiction of yet another embodiment of the subject segmented electrode structures in which electrical connections are provided by coils. In the embodiment depicted in these figures, a coiled spring is provided to attach and provide electrical communication between the IC and one or more elongated conductive members. Compression and stretching forces in the directions of the length of elongated conductive members as in relation to the chip-electrode assembly can lead to strain on attachment to the chip. The use of a spring provides a source of relief for this tension, limiting the strain on the connection. In some cases, the spring may be tapered, providing a graduated transition of the strain. This will limit the impact of a strain in that dimension on the attachments.

In one embodiment of the present invention, a flexible spring is used to provide stress reduction on electrical connections. The spring can be made from many appropriate materials, including but not limited to: platinum, platinum iridium, platinum nickel, platinum tungsten, MP35N, Elgiloy, L605, 316 stainless steel, titanium, nickel titanium, Nitinol, cobalt chromium, cobalt, NiTi, tantalum, among other appropriate material choices.

The flexible spring of the present invention is provided at a length most appropriate to the particular miniaturized device and its application. This can potentially be as long as the device of which it is a part. By example, the length of the spring can be about 0.080 to about 0.200 inches, such as from about 0.030 to about 0.100 inches, and including from about 0.015 to about 0.250 inches. The wire diameter of the spring will be selected as appropriate to the material and as to the particular application. Wire diameter ranges for some embodiments of the present invention are about 0.0005 to about 0.020 inches, such as from about 0.002 to about 0.010 inches, and including about 0.003 inches.

Pressures on the device may also occur as the elongated conductive members curve away from or curve towards the electrode, e.g., quadrant electrode, assembly in either a sideways or up and down directions. These compression and extension forces again can be relieved by the use of the inventive flexible attachment structure, and other stress relief features working synergistically to more rigid structures of the device.

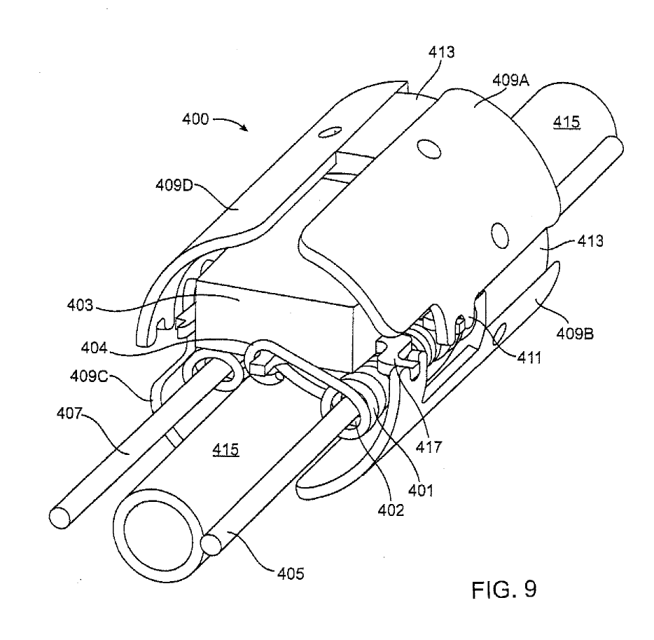

FIG. 9 shows an assembly 400 with flexible connections, in this case, a micro-spring used as part of the assembly. Various other flexible connectors can be employed, as desired. As shown in FIG. 9 flexible connections 401 are provided between IC 403 and elongated conductive members 405 and 407. This design creates a flexible connection between the IC and the elongated conductive members. In this design embodiment, the elongated conductive members 405 and 407 are placed into inner lumen 402 of flexible connections 401, as shown in the assembly.

IC 403 is attached to quadrant electrodes 409A, 409B, 409C and 409D by junctures 411. Quadrant electrodes 409A, 409B, 409C and 409D are joined together with PEEK material 413. Guide wire lumen 415 runs beneath IC 403 and beneath and/or between elongated conductive members 405 and 407, all running through or contained with quadrant electrodes 409A, 409B, 409C and 409D.

The device shown in FIG. 9 enjoys many advantages provided by its various parts and features. By example, the flexible connections 401 provide a fault resistant connection, even in a highly challenging environment such as the heart. The PEEK material 413 joining quadrant electrodes 409 provides structural stability, especially during the subassembly joining. These design innovations assure fatigue resistance and stress reduction of the device without compromising its structural integrity.

Working synergistically with the more fatigue resistant members of the construct, joined areas, such as junctures 411 which can include welding, providing a basic, strong architectural integrity to the device. Such features as attachment tabs 417 assure that these joined portions of the device are well aligned, and also provide additional structural stability, decreasing strain on the weld junctures.

Devices and Systems

Aspects of the invention include devices and systems, including implantable medical devices and systems, that include the hermetically sealed structures according to embodiments of the invention. The devices and systems may perform a number of different functions, including but not limited to electrical stimulation applications, e.g., for medical purposes, analyte, e.g., glucose detection, etc.

The implantable medical devices and system may have a number of different components or elements in addition to the electrodes, where such elements may include, but are not limited to: sensors (e.g., cardiac wall movement sensors, such as wall movement timing sensors); processing elements, e.g., for controlling timing of cardiac stimulation, e.g., in response to a signal from one or more sensors; telemetric transmitters, e.g., for telemetrically exchanging information between the implantable medical device and a location outside the body; drug delivery elements, etc. As such, the subject hermetically sealed structures may be operably coupled, e.g., in electrical communication with, components of a number of different types of implantable medical devices and system, where such devices and systems include, but are not limited to: physiological parameter sensing devices; electrical (e.g., cardiac) stimulation devices, etc.

In certain embodiments of the subject systems and devices, one or more segmented electrode structures of the invention are electrically coupled to at least one elongated conductive member, e.g., an elongated conductive member present in a lead, such as a cardiovascular lead. In certain embodiments, the elongated conductive member is part of a multiplex lead, e.g., as described in Published PCT Application No. WO 2004/052182 and U.S. patent application Ser. No. 10/734,490, the disclosure of which is herein incorporated by reference. In some embodiments of the invention, the devices and systems may include onboard logic circuitry or a processor, e.g., present in a central control unit, such as a pacemaker can. In these embodiments, the central control unit may be electrically coupled to one or more hermetically sealed structures via one or more conductive members.

Devices and systems in which the subject segmented electrode structures find use include, but are not limited to, those described in: WO 2004/066817 titled “Methods And Systems For Measuring Cardiac Parameters”; WO 2004/066814 titled “Method And System For Remote Hemodynamic Monitoring”; WO 2005/058133 titled “Implantable Pressure Sensors”; WO 2004/052182 titled “Monitoring And Treating Hemodynamic Parameters”; WO 2004/067081 titled “Methods And Apparatus For Enhancing Cardiac Pacing”; U.S. Provisional Patent Application 60/638,928 entitled “Methods and Systems for Programming and Controlling a Cardiac Pacing Device” filed Dec. 23, 2004; U.S. Provisional Patent Application No. 60/658,445 titled “Fiberoptic Cardiac Wall Motion Timer” filed Mar. 3, 2005; U.S. Provisional Patent Application No. 60,667,759 titled “Cardiac Motion Detection Using Fiberoptic Strain Gauges,” filed Mar. 31, 2005; U.S. Provisional Patent Application No. 60/679,625 titled “de Minimus Control Circuit for Cardiac pacing and Signal Collection,” filed May 9, 2005; U.S. Provisional Patent Application No. 60/706,641 titled “Deployable Epicardial Electrode and Sensor Array,” filed Aug. 8, 2005; U.S. Provisional Patent Application No. 60/705,900 titled “Electrical Tomography” filed Aug. 5, 2005; U.S. Provisional Patent Application No. 60/707,995 (attorney docket no. PRO-P37) titled “Methods and Apparatus for Tissue Activation and Monitoring” filed Aug. 12, 2005; U.S. Provisional Patent Application No. 60/707,913 titled “Measuring Conduction Velocity Using One or More Satellite Devices,” filed Aug. 12, 2005. These applications are herein incorporated into the present application by reference in their entirety.

Some of the present inventors have developed Doppler, pressure sensors, additional wall motion, and other cardiac parameter sensing devices, which devices or at least components thereof can be present in medical devices according to embodiments of the invention, as desired. Some of these are embodied in currently filed provisional applications; “One Wire Medical Monitoring and Treating Devices”, U.S. Provisional Patent Application No. 60/607,280 filed Sep. 2, 2004, U.S. patent Applications No. 11/025,876 titled “Pressure Sensors having Stable Gauge Transducers”; U.S. patent application Ser. No. 11/025,366 “Pressure Sensor Circuits”; U.S. patent application Ser. No. 11/025,879 titled “Pressure Sensors Having Transducers Positioned to Provide for Low Drift”; U.S. patent application Ser. No. 11/025,795 titled “Pressure Sensors Having Neutral Plane Positioned Transducers”; U.S. patent Application Ser. No. 11/025,657 titled “Implantable Pressure Sensors”; U.S. patent application Ser. No. 11/025,793 titled “Pressure Sensors Having Spacer Mounted Transducers”; “Stable Micromachined Sensors” U.S. Provisional Patent Application 60/615,117 filed Sep. 30, 2004, “Amplified Complaint Force Pressure Sensors” U.S. Provisional Patent Application No. 60/616,706 filed Oct. 6, 2004, “Cardiac Motion Characterization by Strain Measurement” U.S. Provisional Patent Application filed Dec. 20, 2004, and PCT Patent Application entitled “Implantable Pressure Sensors” filed Dec. 10, 2004, “Shaped Computer Chips with Electrodes for Medical Devices” U.S. Provisional Patent Application filed Feb. 22, 2005; “Fiberoptic Cardiac Wall Motion Timer” U.S. Provisional Patent Application 60/658,445 filed Mar. 3, 2005; “Cardiac Motion Detection Using Fiberoptic Strain Gauges” U.S. Provisional Patent Application 60/667,749 filed Mar. 31, 2005. These applications are incorporated in their entirety by reference herein.

In certain embodiments, the implantable medical devices and systems which include the subject segmented electrode structures are ones that are employed for cardiovascular applications, e.g., pacing applications, cardiac resynchronization therapy applications, etc.

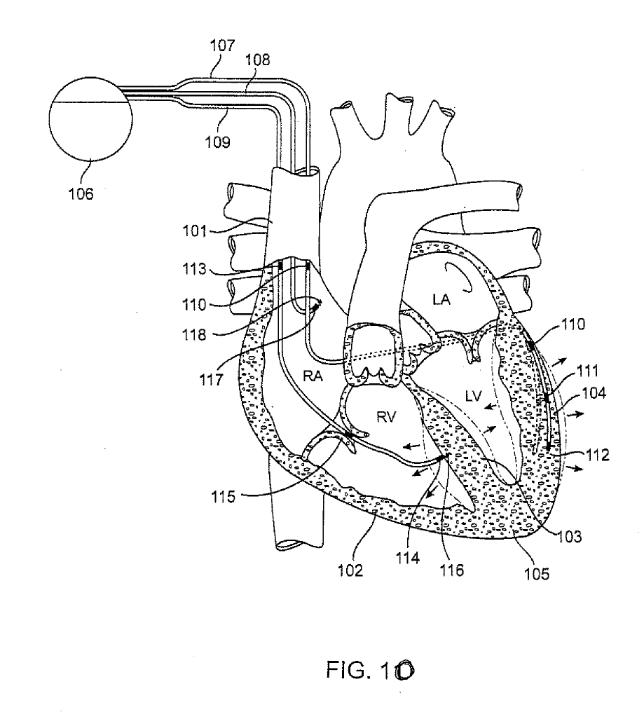

A representative system in which the hermetically sealed integrated structures find use is depicted in FIG. 10, which provides a cross-sectional view of the heart with of an embodiment of a cardiac resynchronization therapy (CRT) system that includes hermetically sealed integrated circuits according to embodiments of the invention. The system includes a pacemaker can 106, a right ventricle electrode lead 109, a right atrium electrode lead 108, and a left ventricle cardiac vein lead 107. Also shown are the right ventricle lateral wall 102, interventricular septal wall 103, apex of the heart 105, and a cardiac vein on the left ventricle lateral wall 104.

The left ventricle electrode lead 107 is comprised of a lead body and one or more electrode assemblies 110,111, and 112. Each of the electrodes includes a hermetically sealed integrated circuit. Having multiple distal electrode assemblies allows a choice of optimal electrode location for CRT. In a representative embodiment, electrode lead 107 is constructed with the standard materials for a cardiac lead such as silicone or polyurethane for the lead body, and MP35N for the coiled or stranded conductors connected to Pt—Ir (90% platinum, 10% iridium) electrode assemblies 110,111 and 112. Alternatively, these device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”, and 20040193021 titled “Method and system for monitoring and treating hemodynamic parameters; the disclosures of which are herein incorporated by reference), to the proximal end of electrode lead 107. The proximal end of electrode lead 107 connects to a pacemaker 106.

The electrode lead 107 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The electrode lead 107 is slid over the guidewire into the left ventricle cardiac vein 104 and tested until an optimal location for CRT is found. Once implanted a multi electrode lead 107 still allows for continuous readjustments of the optimal electrode location.

The electrode lead 109 is placed in the right ventricle of the heart with an active fixation helix at the end 116 which is embedded into the cardiac septum. In this view, the electrode lead 109 is provided with one or multiple electrodes 113,114,115.

Electrode lead 109 is placed in the heart in a procedure similar to the typical placement procedures for cardiac right ventricle leads. Electrode lead 109 is placed in the heart using the standard cardiac lead devices which include introducers, guide catheters, guidewires, and/or stylets. Electrode lead 109 is inserted into the clavicle vein, through the superior vena cava, through the right atrium and down into the right ventricle. Electrode lead 109 is positioned under fluoroscopy into the location the clinician has determined is clinically optimal and logistically practical for fixating the electrode lead 109. Under fluoroscopy, the active fixation helix 116 is advanced and screwed into the cardiac tissue to secure electrode lead 109 onto the septum. The electrode lead 108 is placed in the right atrium using an active fixation helix 118. The distal tip electrode 118 is used to both provide pacing and motion sensing of the right atrium.

Kits

Also provided are kits that include the subject segmented electrode structures, as part of one or more components of an implantable device or system, such as the devices and systems reviewed above. In certain embodiments, the kits further include at least a control unit, e.g., in the form of a pacemaker can. In certain of these embodiments, the structure and control unit may be electrically coupled by an elongated conductive member. In certain embodiments, the segmented electrode sealed structure may be present in a lead, such as a cardiovascular lead.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

The following examples are offered by way of illustration and not by way of limitation.

### EXPERIMENTAL

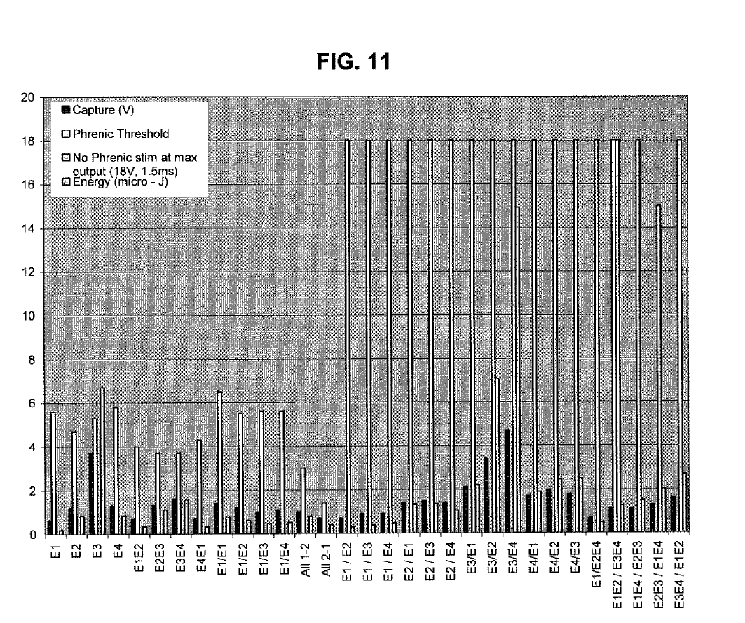

With a standard ring to ring pacing configuration, it is possible to get Phrenic capture thresholds that are almost as low as pacing capture. Some of the present inventors demonstrated this in the animal study and it is shown in the data provided in FIG. 11. The table so provided shows experimental data of Phrenic capture which is 20× greater than the cardiac capture threshold. Notice the two data points on the chart labeled (all 1-2) and (all 2-1).

By switching to a bipolar configuration on a single band (ring location), we were able to significantly raise the Phrenic capture threshold without effecting the cardiac capture threshold. The area on the chart with the tall yellow bars shows this phenomena in action. In each of these arrangements, the cathode and anode electrodes were on the same band or ring.

For this particular band we were able to create a situation where the Phrenic capture is 20× greater than the cardiac capture threshold. Since we were unable to pace at higher voltages, it is expected that the voltage could be considerably higher. In clinical practice, the voltage can range from about 5-50 volts, more specification from about 10-25 volts, and most specifically about 18 volts. A 20× safety factor is well within the usable range. Standard industry practice is to test for capture of the Phrenic nerve at 10V. If there is no Phrenic capture at this voltage, the location is considered good from that perspective.

The data combined with the pictures we took verifies the directionality of the pacing pulse and our ability to be selective about which tissue we are capturing with the pacing pulse. The quad electrodes give us a high level of “Selectivity”. For instance, heart muscle capture ranges from about 0.25 to 10 volts, specifically from about 0.50 to 5 volts, and most specifically about 1.5 volts.

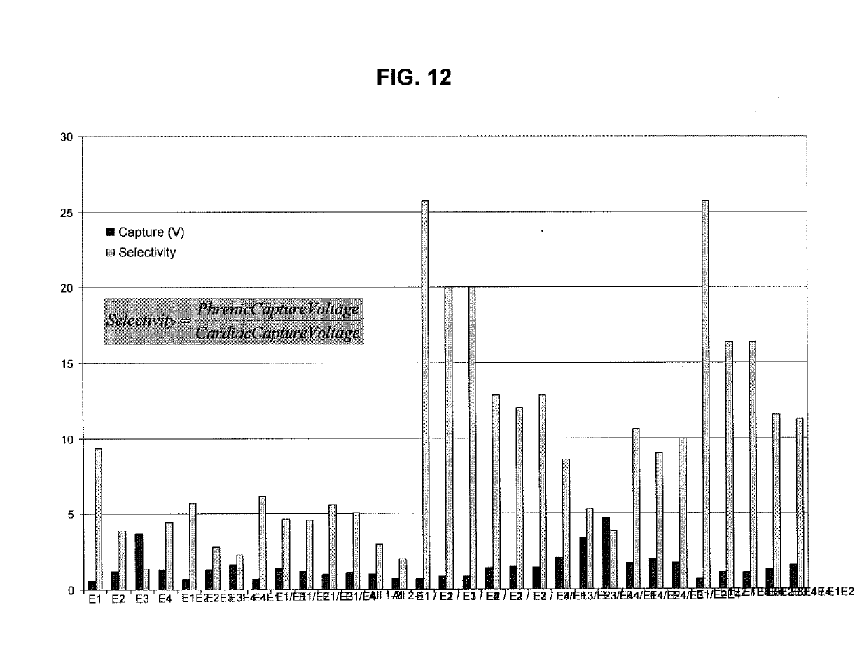

Another way to consider the data provided in FIG. 11 is shown in FIG. 12. This introduces the concept of Selectivity which is the ratio of the phrenic nerve capture voltage to the cardiac capture voltage, In this case, the larger the number, the greater the clinical benefit. This view of the data shows just two variables; capture voltage and selectivity. While simpler than the 4-parameter table in FIG. 11, it provides a simple, direct understanding of the clinical significance of the present invention.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method comprising:
implanting an implantable medical device have an electrical tissue stimulation element into a subject; and
activating said electrical tissue stimulation element in a manner sufficient to stimulation tissue with high selectivity.

2. The method according to claim 1, wherein said tissue is cardiac tissue.

3. The method according to claim 2, wherein said method is a method of stimulating cardiac tissue in a manner that has a high phrenic nerve capture threshold.

4. The method according to claim 3, wherein said method has a low cardiac tissue capture threshold.

5. The method according to claim 2, wherein said method includes obtaining phrenic nerve capture data.

6. The method according to claim 5, wherein said obtaining of said phrenic nerve capture data comprises employing a sensor.

7. The method according to claim 5, wherein said obtaining of said phrenic nerve capture data comprises employing non-cardiac pacing pulses.

8. The method according to claim 5, wherein said method further comprises employing said obtained phrenic nerve capture data in determining a cardiac pacing protocol.

9. The method according to claim 2, wherein said method comprises employing a segmented electrode structure that includes two or more distinct electrode elements.

10. The method according to claim 9, wherein said method comprises activating at least one of said electrodes of said structure to deliver electrical energy to said subject.

11. The method according to claim 10, wherein at least a first of said electrodes is connected to a first conductive member and a second of said electrodes is connected to a second conductive member.

12. The method according to claim 10, wherein said method comprises not activating at least one of said electrodes.

13. The method according to claim 10, wherein said method further comprises determining which of said electrodes to activate.

14. The method according to claim 10, wherein said method comprises activating said electrodes in manner sufficient to not stimulate the phrenic nerve.

15. A system comprising:
at least one implantable lead comprising a segmented electrode; and
a control unit configured to operate said lead according to a method of claim 1.

16. A kit comprising:
at least one implantable lead comprising a segmented electrode; and
a control unit configured to operate said lead according to a method of claim 1.
